(19) United States
(12) Patent Application Publication
(10) Pub. No.: US 2010/0056519 A1
(43) Pub. Date: Mar. 4, 2010

(54) COMPOSITION AND METHOD FOR REDUCING PLATELET ACTIVATION AND FOR THE TREATMENT OF THROMBOTIC EVENTS

(76) Inventors: Victor L. Serebruany, West Friendship, MD (US); Daniela Dastros-Pitei, London (GB); Motoji Kogushi, Tokyo (JP)

Correspondence Address: DICKSTEIN SHAPIRO LLP, 1633 Broadway, NEW YORK, NY, 10019, US

(21) Appl. No.: 12/502,899

(22) Filed: Jul. 14, 2009

Related U.S. Application Data
(60) Provisional application No. 61/080,791, filed on Jul. 15, 2008.

(51) Int. Cl. A61K 31/5377; C12N 5/02

(52) U.S. Cl. 514/235.2; 435/375

(57) ABSTRACT

A novel method of treating and preventing a vascular disease in an individual, comprising: selecting an individual having an elevated level of glycoprotein Ib, glycoprotein IIb/IIIa, PECAM-1, vitronectin receptor or thrombospondin receptor, or formation of platelet-neutrophil aggregates; and administering a therapeutically effective amount of a protease activated receptor-1 (PAR-1) antagonist, a pharmaceutical salt thereof, or a solvate thereof to the individual; and also, composition for treating cardiovascular diseases.

### RELATED APPLICATION

This application claims priority under 35 U.S.C. §119 to U.S. Provisional Application No. 61/080,791, filed Jul. 15, 2008, the disclosure of which is incorporated by reference in its entirety.

### FIELD OF THE INVENTION

The invention relates to treating or preventing a vascular disease.

### BACKGROUND

Protease-activated receptor-1 (PAR-1) is a G protein-coupled receptor which modulates thrombin-platelet interactions. E5555 is a small molecule that acts as a PAR-1 antagonist. The drug shows promises in prevention of thrombotic vascular events, and is being tested in Phase II trials in patients with coronary artery disease (CAD). Since increased platelet activation and thrombin generation has been linked to occlusive outcomes, we investigated the in vitro effects of E5555 on platelet function beyond PAR-1 blockade in healthy volunteers and CAD patients treated with aspirin (ASA), with or without clopidogrel.

### SUMMARY OF THE INVENTION

The inventors have found that administration of a PAR-1 antagonist provides a treatment effect for vascular disease.

A method of treating a vascular disease in an individual having an elevated level of glycoprotein Ib, glycoprotein IIb/IIIa, PECAM-1, vitronectin receptor or thrombospondin receptor, or formation of platelet-neutrophil aggregates by administering a therapeutically effective amount of a PAR-1 antagonist, a pharmaceutical salt thereof or a solvate thereof, to the individual.

The above methods, wherein the individual is also selected for treatment based on the individual having a PAR-1 platelet expression in a normal range.

The above methods, wherein the vascular disease treated can be coronary artery disease, myocardial infarction, angina, stroke, pulmonary embolism, transient ischemic attack, deep vein thrombosis, thrombotic re-occlusion subsequent to a coronary intervention procedure, heart surgery, vascular surgery, peripheral vascular thrombosis, Syndrome X, heart failure, and a disease in which a narrowing of at least one coronary artery occurs.

The above methods, wherein the PAR-1 antagonist is 1-(3-tert-butyl-4-methoxy-5-morpholinophenyl)-2-(5,6-diethoxy-7-fluoro-1-imino-1,3-dihy dro-2H-isoindol-2-yl)ethanone hydrobromide or a metabolite thereof.

The above methods, wherein the PAR-1 antagonist is administered orally in a therapeutically effective amount that is between about 0.1 mg and about 500 mg per day.

The above methods, wherein the elevated level of PAR-1, glycoprotein Ib, glycoprotein IIb/IIIa, PECAM-1, vitronectin receptor or thrombospondin receptor, or formation of neutrophil aggregates is found on platelets.

A method of treating vascular disease by assessing a level of glycoprotein Ib, glycoprotein IIb/IIIa, PECAM-1, vitronectin receptor or thrombospondin receptor, or formation of platelet-neutrophil aggregates in the individual, and comparing that level or formation of said aggregates to a control to determine if an elevated level exists. When an elevated level of glycoprotein Ib, glycoprotein IIb/IIIa, PECAM-1, vitronectin receptor or thrombospondin receptor, or formation of platelet-neutrophil aggregates exists, administering a therapeutically effective amount of a PAR-1 antagonist, a pharmaceutical salt thereof, or a solvate thereof to the individual to reduce the level of glycoprotein Ib, glycoprotein Ib/IIIa, PECAM-1, vitronectin receptor or thrombospondin receptor, or formation of platelet-neutrophil aggregates to treat the vascular disease.

The above methods, further including reducing the level of glycoprotein Ib, glycoprotein IIb/IIIa, PECAM-1, vitronectin receptor or thrombospondin receptor, or formation of platelet-neutrophil aggregates by at least 10%, as compared to the assessed level of glycoprotein Ib, glycoprotein IIb/IIIa, PECAM-1, vitronectin receptor or thrombospondin receptor, or assessed formation of platelet-neutrophil aggregates.

The above methods, wherein the vascular disease can be coronary artery disease, myocardial infarction, angina, stroke, pulmonary embolism, transient ischemic attack, deep vein thrombosis, thrombotic re-occlusion subsequent to a coronary intervention procedure, heart surgery, vascular surgery, peripheral vascular thrombosis, Syndrome X, heart failure, and a disease in which a narrowing of at least one coronary artery occurs.

The above methods, including administering one or more of vascular disease treating drugs.

The above methods, wherein the PAR-1 antagonist is 1-(3-tert-butyl-4-methoxy-5-morpholinophenyl)-2-(5,6-diethoxy-7-fluoro-1-imino-1,3-dihy dro-2H-isoindol-2-yl)ethanone hydrobromide or a metabolite thereof.

A method of preventing a vascular disease in an individual having an elevated level of glycoprotein Ib, glycoprotein IIb/IIIa, PECAM-1, vitronectin receptor or thrombospondin receptor, or formation of platelet-neutrophil aggregates by administering a therapeutically effective amount of a PAR-1 antagonist, a pharmaceutical salt thereof, or a solvate thereof to the individual.

A method of preventing a vascular disease in an individual by assessing a level of glycoprotein Ib, glycoprotein IIb/IIIa, PECAM-1, vitronectin receptor or thrombospondin receptor, or formation of platelet-neutrophil aggregates in the individual, comparing that level or formation of aggregates to a control to determine if an elevated level of glycoprotein Ib, glycoprotein IIb/IIIa, PECAM-1, vitronectin receptor or thrombospondin receptor, or formation of platelet-neutrophil aggregates exists; and, when an elevated level of glycoprotein Ib, glycoprotein IIb/IIIa, PECAM-1, vitronectin receptor or thrombospondin receptor, or formation of platelet-neutrophil aggregates exists, administering a therapeutically effective amount of a PAR-1 antagonist, a pharmaceutical salt thereof, or a solvate thereof to the individual.

A method of inhibiting the production of glycoprotein Ib, glycoprotein IIb/IIIa, PECAM-1, vitronectin receptor or thrombospondin receptor, or formation of platelet-neutrophil aggregates on a platelet in an individual at risk for developing a vascular disease by administering to the individual a therapeutically effective amount of a PAR-1 antagonist, a pharmaceutical salt thereof or a solvate thereof.

The above methods, wherein the platelet is contacted with the PAR-1 antagonist, a pharmaceutical salt thereof, or a solvate thereof ex vivo.

Any of the above methods wherein the PAR-1 antagonist is administered orally, intravenously, intramuscularly, subcutaneously, parenterally, nasally, by inhalation, by implant, or by suppository.

Any of the above methods wherein the method also includes administering a second compound selected from the group consisting of an anti-platelet compound, an antithrombotic, an anticoagulant, or a thrombolyic agent.

Any of the above methods wherein the individual is a human.

### BRIEF DESCRIPTION OF THE DRAWINGS

FIG. 1 is a view showing PAR-1 activation mechanisms by thrombin.

### DETAILED DESCRIPTION OF THE INVENTION

The following embodiments are illustration of the present invention and are not intended to limit the invention. In the following detailed description embodiments are described in sufficient detail to enable those of ordinary skill in the art to make and use the invention, and it should be understood that structural, logical, or procedural changes may be made to the specific embodiments disclosed without departing from the invention. When referring to the FIGURE, use of like reference numbers refer to like features. Publications cited are incorporated herein in their entirety.

A PAR-1 antagonist is effective in treatment of coronary artery disease and displays an inhibitory effect on platelet function. The invention is provides methods of treating vascular diseases.

The invention provides a method of treating a vascular disease in an individual by selecting an individual having an elevated level of glycoprotein Ib, glycoprotein IIb/IIIa, PECAM-1, vitronectin receptor or thrombospondin receptor, or formation of platelet-neutrophil aggregates; and administering a therapeutically effective amount of a PAR-1 antagonist, a pharmaceutical salt thereof, or a solvate thereof to the individual.

This method may also be performed for prophylactic purpose. Therefore, the invention also provides a method of preventing a vascular disease in an individual, comprising selecting an individual having an elevated level of glycoprotein Ib, glycoprotein IIb/IIIa, PECAM-1, vitronectin receptor or thrombospondin receptor, or formation of platelet-neutrophil aggregates; and administering a therapeutically effective amount of a PAR-1 antagonist, a pharmaceutical salt thereof, or a solvate thereof to the individual.

Without intending to limit the scope of the invention, the following definitions are provided:

As used herein, “a vascular disease” refers to any disease or disorder which primarily affects the blood vessels. As used herein, “disease” and “disorder” are used interchangeably. Examples of vascular disease include coronary artery disease, myocardial infarction, angina, stroke, pulmonary embolism, transient ischemic attack, deep vein thrombosis, thrombotic re-occlusion subsequent to a coronary intervention procedure, heart surgery, vascular surgery, peripheral vascular thrombosis, Syndrome X, heart failure, and a disease in which a narrowing of at least one coronary artery occurs.

As used herein, “glycoprotein Ib” is a component of the glycoprotein Ib-V-IX complex on platelets and also referred to as “CD42” (Bode A P, Read M S, Reddick R L, J. Lab. Clin. Med. 133 (2): 200-11. (February 1999)).

As used herein, “glycoprotein Ib/IIIa” is an integrin found on platelets. It is a receptor for fibrinogen and aids in platelet activation. The complex is formed via calcium-dependent association of gpIIb and gpIIIa, a required step in normal platelet aggregation and endothelial adherence as explained by Calvete J J, Proc. Soc. Exp. Biol. Med. 208 (4): 346-60 (1995) and Shattil S J, Thromb. Haemost. 82 (2): 318-25 (1999).

As used herein, “PECAM-1” is the abbreviation of platelet endothelial cell adhesion molecule-1 and also referred to as “CD31.” PECAM-1 plays a key role in removing aged neutrophils from the body and is normally found on endothelial cells, platelets, macrophages and Kupffer cells, granulocytes, T/NK cells, lymphocytes, megakaryocytes, osteoclasts and neutrophils.

As used herein, “vitronectin receptor” is a member of the integrin superfamily of adhesive protein receptors that mediate a wide spectrum of adhesive cellular interactions, including attachment to vitronectin, von Willebrand factor, fibrinogen, and thrombospondin as explained by Charo I F et al, JCB, 111(6): 2795-2800 (1990).

As used herein, “thrombospondin receptor” is a protein also referred to as “CD36” that can bind to thrombospondin, a protein which plays roles in platelet aggregation, angiogenesis, and tumorigenesis as explained by Silverstein R L et al., J. Biol. Chem., 267(23):16607-16612, 08, (1992) and Entrez Gene: THBS1 thrombospondin 1.

The level of glycoprotein Ib, glycoprotein IIb/IIIa, PECAM-1, vitronectin receptor or thrombospondin receptor can be measured by conventional methods such as flow-cytometry and enzyme liked immunosorbent assay (ELISA) using specific antibodies against these proteins.

“Platelet-neutrophil aggregates” are formed through interactions between platelets and neutrophils. Neutrophil-platelet aggregation may occur in the circulation and be pathophysiologically significant. Enhanced neutrophil-platelet adhesion has been observed in the circulation of patients with vascular diseases such as acute myocardial infarction (AMI) or stroke and after coronary angioplasty as explained in Circulation. 1998; 98:873-882. Platelet-neutrophil aggregates can be determined using a Coulter Counter ZM as described herein.

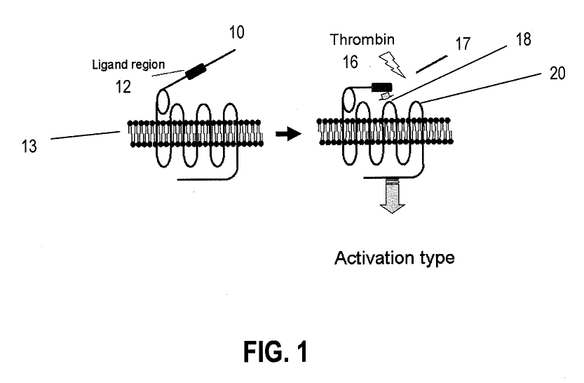

“Protease-activated receptor-1 (PAR-1)” is a protease-activated receptors and is a G protein-coupled receptor that is activated due to degradation of a particular extracellular region by protease. The activation mechanism of PAR-1 10 is shown in FIG. 1 acting at cell membrane 13. Activation of the receptor 20 occurs when a certain N-terminal site 17 of the receptor 20 is cleaved with serine protease 16 to expose the receptor-activating sequence 12, which acts as a ligand and binds to the ligand binding site 18 of the receptor 20.

The elevated level of PAR-1, glycoprotein Ib, glycoprotein IIb/IIIa, PECAM-1, vitronectin receptor or thrombospondin receptor, or formation of neutrophil aggregates is found on platelets.

As used herein, “individuals” refers to animals, preferably mammals, more preferably humans. In an embodiment, the individual may be one in healthy condition or a patient with one or more of vascular diseases such as coronary artery disease, and more preferably, a patient with coronary artery disease currently taking vascular disease treating drugs such as aspirin, clopidogrel, or the combination thereof.

As used herein, “elevated level” means a level which is higher than a normal level considered harmless to an individual's health.

As used herein, “treatment” and “treating” refer to the acquisition of a desired pharmacological effect and/or physiologic effect. These effects are prophylactic in terms of completely or partially preventing a disease and/or symptom(s), and therapeutic in terms of partially or completely curing a disease and/or an adverse event caused by a disease. “Treatment” and “treating” include any treatment of a disease in a patient including, for example: (a) to prevent a disease or symptom(s) in a patient who is suspected of being predisposed to the disease or symptom(s) but is not yet diagnosed to be so; (b) to inhibit a symptom(s) of a disease, i.e., to inhibit or delay the progression of the symptom(s); and (c) to alleviate a symptom(s) of a disease, i.e., to reverse or eliminate the symptom(s) of the disease; or to reverse the progress of the symptom(s). Specifically, treatment of a vascular disease includes reducing the level of glycoprotein Ib, glycoprotein IIb/IIIa, PECAM-1, vitronectin receptor or thrombospondin receptor, or the amount of platelet-neutrophil aggregates in the subject, preferably, by about 10% or more, as compared to the level of glycoprotein Ib, glycoprotein IIb/IIIa, PECAM-1, vitronectin receptor or thrombospondin receptor, or the amount of platelet-neutrophil aggregates having been measured prior to administration of the PAR-1 antagonist.

As used herein, “preventing a vascular disease” refers to preventing the onset or further progression of a clinical symptom(s) and/or a pathological sign(s) of a vascular disease. For example, progression of a clinical symptom or a pathological sign can be prevented for patients who do not exhibit the clinical symptom(s) or pathological sign(s) of a vascular disease. In addition, patients with a milder form of a vascular disease can be prevented from progressing to a more severe form of the vascular disease. Specifically, prevention of a vascular disease includes maintaining a normal level of glycoprotein Ib, glycoprotein IIb/IIIa, PECAM-1, vitronectin receptor or thrombospondin receptor, or a normal amount of platelet-neutrophil aggregates in the subject, or preventing the increase in the level of glycoprotein Ib, glycoprotein IIb/IIIa, PECAM-1, vitronectin receptor or thrombospondin receptor, or in the amount of platelet-neutrophil aggregates.

As used herein, “PAR-1 antagonist” is not limited as long as it is a substance that binds to PAR-1 and that inhibits binding between a polypeptide part containing a receptor-activating sequence and PAR-1. Examples of preferable properties of the PAR-1 antagonist include treating effects on vascular diseases without cause severe side-effects (such as further bleeding) at a therapeutically effective amount.

The PAR-1 antagonist can be 1-(3-tert-butyl-4-methoxy-5-morpholinophenyl)-2-(5,6-diethoxy-7-fluoro-1-imino-1,3-dihy dro-2H-isoindol-2-yl)ethanone hydrobromide or a metabolite thereof. This compound, is also known as “E5555” and has the following formula:

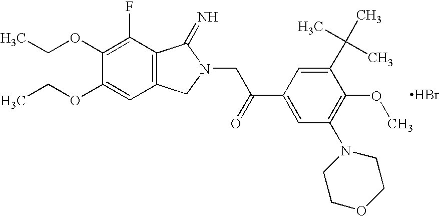

This compound has a molecular weight of 608.54 as the bromide salt. The PAR-1 antagonist may be in the form of a pharmaceutically acceptable salt thereof, or a solvate thereof.

Herein, “pharmaceutically acceptable salt” is not limited as long as it has an effect of antagonistic function of PAR-1 and forms a pharmaceutically acceptable salt with a compound of the invention in terms of a therapeutic agent for vascular diseases. Specifically, examples include, but not limited to, hydrohalide (e.g., hydrofluoride, hydrochloride, hydrobromide, hydroiodide, etc.), salts of inorganic acids (e.g., sulfate, nitrate, perchlorate, phosphate, carbonate, bicarbonate, etc.), organic carboxylates (e.g., acetate, oxalate, maleate, tartrate, fumarate, citrate, etc.), organic sulfonates (e.g., methanesulfonate, trifluoromethanesulfonate, ethanesulfonate, benzenesulfonate, toluenesulfonate, camphorsulfonate, etc.), amino acid salts (e.g., aspartate, glutamate, etc.), quaternary amine salts, alkali metal salts (e.g., sodium salt, potassium salt, etc.) and alkaline earth metal salts (e.g., magnesium salt, calcium salt, etc.).

“Solvate” includes hydrate. The PAR-1 antagonist may be in anhydride form, and may form a solvate such as a hydrate. The solvate may be either a hydrate or a nonhydrate, preferably a hydrate. The solvent used may be water, alcohol (e.g., methanol, ethanol or n-propanol), dimethylformamide or the like.

“Hydrate” refers to an active ingredient or compound containing a crystallized molecule of water. The molecule of water of crystallization can be an integer of 1 or more, such as 1 to 10; or can be any fraction greater than 0 or a fraction of an integer from 1 to 10. For example, the hydrate may be represented as (active ingredient)·¼H₂O; (active ingredient)·½H₂O; (active ingredient)·¾H₂O; (active ingredient)·2H₂O; (active ingredient)·5½H₂O; (active ingredient)·6H₂O; and the like.

The administration mode of the PAR-1 antagonist is not particularly limited and they may be administrated either orally or parenterally. Specific examples of administration include oral, intravenous, intramuscular, subcutaneous, parenteral and nasal administrations, and administrations by inhalation, by implant, and by suppository, for example. Preferably, the PAR-1 antagonist is administered orally.

Examples of modes for parenteral administration include intravenous injection, drip infusion, subcutaneous injection, intradermal injection, intrathecal injection and intraperitoneal injection. Examples of formulations include tablets, powdered agents, subtle granules, granules, capsules and syrup for oral administration modes, and suppositories, injectable agents, ointments, and skin patches for parenteral administration modes.

When preparing an oral formulation used for oral administration mode, an excipient, and if necessary, a binder, a disintegrant, a lubricant, a colorant, a flavoring agent, or the like are added to the active ingredient, and then formulated into a tablet, a coated tablet, granule, subtle granule, powdered agent, a capsule, or the like by a conventional procedure.

As an excipient, for example, lactose, cornstarch, sucrose, glucose, sorbit, crystalline cellulose, silicon dioxide or the like may be used. As a binder, for example, polyvinyl alcohol, ethyl cellulose, methyl cellulose, gum arabic, hydroxypropyl cellulose, hydroxypropyl methyl cellulose, or the like may be used. As a lubricant, for example, magnesium stearate, talc, silica or the like may be used. As a colorant, those that are acceptable as an additive to a pharmaceutical product may be used. As a flavoring agent, cocoa powder, menthol, aromatic acid, mint oil, camphor, cinnamon powder or the like may be used. Of course, these tablets and granules may conveniently be coated with sugar, gelatin, or the like, if necessary.

According to the present invention, an injectable agent may be prepared by adding, if necessary, a nonaqueous dilution agent (e.g., glycols such as propylene glycol and polyethylene glycol, plant oils such as olive oil and alcohols such as ethanol), a suspending agent, a solubilizing agent, a stabilizer, a tonicity agent, a preservative, a pH regulator, a buffer or the like to the principal agent. Sterilization of the injectable agent may be achieved by filter sterilization, addition of a disinfectant or the like. Furthermore, the injectable agent may be produced in a form that can be prepared upon use. Specifically, the injectable agent can be made into a sterile solid composition by lyophilization, or the like so as to be dissolved in sterile injectable distilled water or other solvent before use. If the agent is to be administered by transdermal absorption via an adhesive patch, it is preferable that the agent that does not form a salt, (i.e., a so-called a free form) is selected. The injectable agent may be made into a drip infusion, or an intravenous, subcutaneous or intramuscular injectable agent according to a conventional procedure.

Examples of suspending agents include methyl cellulose, Polysorbate 80, hydroxyethyl cellulose, gum arabic, powdered tragacanth, sodium carboxymethyl cellulose and polyoxyethylene sorbitan monolaurate.

Examples of solubilizing agents include polyoxyethylene hydrogenated castor oil, Polysorbate 80, nicotinic-acid amide, polyoxyethylene sorbitan monolaurate, macrogol and castor oil fatty acid ethyl ester.

Examples of stabilizers include, for example, sodium bisulfite and sodium metabisulfite.

Examples of preservatives include, for example, methyl parahydroxybenzoate, ethyl parahydroxybenzoate, sorbic acid, phenol, cresol and chlorocresol.

The PAR-1 antagonist may be administered together with one or more of pharmaceutical compositions such as a second compound or vascular disease treating drugs. The second compound is selected from the group consisting of an anti-platelet compound, an anticoagulant or a thrombolyic agent. Preferable examples of the vascular disease treating drugs include aspirine, clopidogrel and the combination thereof.

“Therapeutically effective amount” refers to the amount of the active ingredient, for example, a PAR-1 antagonist, vascular disease treating drugs such as aspirin, clopidogrel or the combination thereof, or a second compound selected from an anti-platelet compound, an anticoagulant or a thrombolyic agent, that is necessary for the treatment and/or prophylaxis and/or delayed onset of a vascular disease(s). When two or more active ingredients are administered as a pharmaceutical composition or for combination therapy, the term “therapeutically effective amount” refers to the amount of active ingredients that are necessary for treatment and/or prophylaxis and/or delayed onset of a disease and includes, for example: (a) a therapeutically effective amount of a first active ingredient and a therapeutically effective amount of a second active ingredient (i.e., the amount of each active ingredient that would be used for monotherapy for the treatment and/or prophylaxis of a disease is used for the pharmaceutical composition or combination therapy); (b) a therapeutically effective amount of a first active ingredient and a sub-therapeutic amount of a second active ingredient, which in combination effectively provide for treatment and/or prophylaxis of a disease (e.g., the sub-therapeutic amount of the second active ingredient can be used in a pharmaceutical composition or combination therapy to achieve a result that would be equal to or greater than the result that the second active ingredient would achieve if it was used for monotherapy); (c) a sub-therapeutic amount of a first active ingredient and a therapeutically effective amount of a second active ingredient, which in combination effectively provide for treatment and/or prophylaxis of a disease (e.g., the sub-therapeutic amount of the first active ingredient can be used in a pharmaceutical composition or combination therapy to achieve a result that would be equal to or greater than the result that the first active ingredient would achieve if it was used for monotherapy); and (d) a sub-therapeutic amount of a first active ingredient and a sub-therapeutic amount of a second active ingredient, which in combination therapy provide for treatment and/or prophylaxis of a disease or disorder (e.g., the sub-therapeutic amount of the first active ingredient can be used in a pharmaceutical composition or combination therapy to achieve a result that would be equal to or greater than the result that the first active ingredient would achieve if it was used for monotherapy; and the sub-therapeutic amount of the second active ingredient can be used in pharmaceutical composition or combination therapy to achieve a result that would be equal to or greater than the result that the second active ingredient would achieve if it was used for monotherapy).

For oral administration, a therapeutically effective amount of the PAR-1 antagonist, its pharmaceutically acceptable salt, or a solvate thereof, differs depending on severity of the symptom, age, sex, weight and sensitivity difference of the patient, administration method, administration time, administration interval, administration period, property, dosage, and type of the active ingredient of the formulation or the like, but may be determined appropriately by those skilled in the art. For example, 0.1-500 mg/day, preferably 0.5-500 mg/day, more preferably 1-500 mg/day, may be orally administered to an average adult (weight about 60 kg).

For parenteral administration of, for example, an injectable agent, a therapeutically effective amount of the PAR-1 antagonist, its pharmaceutically acceptable salt, or a solvate thereof, differs depending on severity of the symptom, age, sex, weight and sensitivity difference of the patient, the administration method, administration time, administration interval, administration period, property, dosage, type and type of active ingredient of the formulation or the like, but may be determined appropriately by those skilled in the art. Such an agent dissolved or suspended in a pharmaceutically acceptable carrier, such as saline or commercially available injectable distilled water, can suitably be given to a patient in need of the treatment. For example, in the case of an injectable agent, 0.1-500 mg/day, preferably 0.5-500 mg/day, more preferably 1-500 mg/day, may be administered to an average adult (weight about 60 kg).

In another embodiment a method of treating a vascular disease in an individual is provided, comprising assessing a level of glycoprotein Ib, glycoprotein IIb/IIIa, PECAM-1, vitronectin receptor or thrombospondin receptor, or formation of platelet-neutrophil aggregates in the individual, and comparing said level or formation of said aggregates to a control to determine if an elevated level of glycoprotein Ib, glycoprotein IIb/IIIa, PECAM-1, vitronectin receptor or thrombospondin receptor, or formation of platelet-neutrophil aggregates exists. If an elevated level of glycoprotein Ib, glycoprotein IIb/IIIa, PECAM-1, vitronectin receptor or thrombospondin receptor, or formation of platelet-neutrophil aggregates is found, then administering a therapeutically effective amount of a PAR-1 antagonist, a pharmaceutical salt thereof, or a solvate thereof, to the individual to reduce the level of glycoprotein Ib, glycoprotein IIb/IIIa, PECAM-1, vitronectin receptor or thrombospondin receptor, or formation of platelet-neutrophil aggregates to treat the vascular disease.

This method may also be performed for prophylactic purpose. Therefore, an embodiment also provides a method of preventing, rather than treating a vascular disease in an individual, comprising assessing a level of glycoprotein Ib, glycoprotein IIb/IIIa, PECAM-1, vitronectin receptor or thrombospondin receptor, or formation of platelet-neutrophil aggregates in the individual, and comparing said level or formation of said aggregates to a control to determine if an elevated level of glycoprotein Ib, glycoprotein IIb/IIIa, PECAM-1, vitronectin receptor or thrombospondin receptor, or formation of platelet-neutrophil aggregates exists. If an elevated level of glycoprotein Ib, glycoprotein IIb/IIIa, PECAM-1, vitronectin receptor or thrombospondin receptor, or formation of platelet-neutrophil aggregates exists, then administering a therapeutically effective amount of a PAR-1 antagonist, a pharmaceutical salt thereof, or a solvate thereof, to the individual.

According to another embodiment, the PAR-1 antagonist is preferably 1-(3-tert-butyl-4-methoxy-5-morpholinophenyl)-2-(5,6-diethoxy-7-fluoro-1-imino-1,3-dihy dro-2H-isoindol-2-yl)ethanone hydrobromide or a metabolite thereof. According to the embodiment, a route and a method for administering the PAR-1 inhibitor are not particularly limited and may refer to the description of the administration and dosage of the invention above. In the embodiment, the vascular disease is preferably coronary artery disease, myocardial infarction, angina, stroke, pulmonary embolism, transient ischemic attack, deep vein thrombosis, thrombotic re-occlusion subsequent to a coronary intervention procedure, heart surgery, vascular surgery, peripheral vascular thrombosis, Syndrome X, heart failure, and a disease in which a narrowing of at least one coronary artery occurs.

As used herein, the term “assessing” means to evaluate or measure the level of vascular disease markers such as glycoprotein Ib, glycoprotein IIb/IIIa, PECAM-1, vitronectin receptor or thrombospondin receptor, or formation of platelet-neutrophil aggregates. As used herein, “assessing,” “measuring,” “evaluating,” and “determining” may be used interchangeably. The levels of glycoprotein Ib, glycoprotein IIb/IIIa, PECAM-1, vitronectin receptor and thrombospondin receptor can readily be evaluated or measured through a variety of techniques known to those who skilled in the art, and some examples of these techniques are described below. Formation of platelet-neutrophil aggregates can be assessed indirectly by measuring the levels of platelet activation markers and/or neutrophil activation markers, or directly by immunohistological observation and the like. Alternatively, the formation of platelet-neutrophil aggregates can readily be determined using a Coulter Counter ZM as described as follows.

As used herein, “control” means the level or value of glycoprotein Ib, glycoprotein IIb/IIIa, PECAM-1, vitronectin receptor or thrombospondin receptor in a healthy individual in which substantially no platelet-neutrophil aggregates being formed.

In some aspects, the method may further comprise reducing the level of glycoprotein Ib, glycoprotein IIb/IIIa, PECAM-1, vitronectin receptor or thrombospondin receptor, or formation of platelet-neutrophil aggregates by at least 10%, as compared to the assessed level of glycoprotein Ib, glycoprotein IIb/IIIa, PECAM-1, vitronectin receptor or thrombospondin receptor, or assessed formation of platelet-neutrophil aggregates.

In still another embodiment, provided is a method of inhibiting the production of glycoprotein Ib, glycoprotein IIb/IIIa, PECAM-1, vitronectin receptor or thrombospondin receptor, or formation of platelet-neutrophil aggregates on a platelet in an individual who is at risk for developing a vascular disease, comprising administering to the individual a therapeutically effective amount of a PAR-1 antagonist, a pharmaceutical salt thereof, or a solvate thereof.

According to this embodiment, the PAR-1 antagonist is preferably 1-(3-tert-butyl-4-methoxy-5-morpholinophenyl)-2-(5,6-diethoxy-7-fluoro-1-imino-1,3-dihy dro-2H-isoindol-2-yl)ethanone hydrobromide or a metabolite thereof.

The route and a method for administering the PAR-1 inhibitor are not particularly limited and may refer to the description of the administration and dosage of the invention above. In the method of the invention, the vascular disease is preferably one of coronary artery disease, myocardial infarction, angina, stroke, pulmonary embolism, transient ischemic attack, deep vein thrombosis, thrombotic re-occlusion subsequent to a coronary intervention procedure, heart surgery, vascular surgery, peripheral vascular thrombosis, Syndrome X, heart failure, and a disease in which a narrowing of at least one coronary artery occurs.

As used herein, “inhibiting” the production of glycoprotein Ib, glycoprotein IIb/IIIa, PECAM-1, vitronectin receptor or thrombospondin receptor, or formation of platelet-neutrophil aggregates on a platelet means to reduce the amount of glycoprotein Ib, glycoprotein IIb/IIIa, PECAM-1, vitronectin receptor or thrombospondin receptor, or the rate of platelet-neutrophil aggregate formation.

“An individual who is at risk for developing a vascular disease” means an individual who is more susceptible to any types of vascular diseases because of the individual's hereditary background, surrounding environment, dietary habit, and life styles. Whether or not a given individual is at risk for developing a vascular disease can be determined by examining the serum lipid composition or by genetic analysis such as gene typing of single nucleotide polymorphisms (SNPs).

Serum lipid composition can be examined using commercially available analysis kit such as Cholesterol/Cholesteryl Ester Quantitation Kit from CALBIOCHEM®, Triglyceride Quantification Kit and HDL and LDL/VLDL Cholesterol Assay Kit from Abcam®. If the individual has a higher serum level of free cholesterol or triglyceride, or higher proportion of low-density lipoprotein against high-density lipoprotein than normal or standard levels, then the individual would be diagnosed to be at risk for developing vascular disease.

Analysis of SNPs can be performed using, e.g., TaqMan® SNP Genotyping Assays. Certain genetic patterns such as the haplotypes in the gene vitamin K epoxide reductase complex subunit 1 (VKORC1) have been reported to be associated with vascular diseases including stroke, coronary heart disease and aortic dissection (Circulation. 2006; 113:1615-1621), hence, can be utilized as genetic markers for determining whether the individual is at risk for developing any vascular disease.

In some cases, the platelet is contacted with the PAR-1 antagonist, a pharmaceutical salt thereof, or a solvate thereof ex vivo.

In another embodiment, the invention relates to a composition for treating or preventing a disease associated with platelet activation by inhibiting glycoprotein Ib, glycoprotein IIb/IIIa, PECAM-1, vitronectin receptor, thrombospondin receptor, and platelet-neutrophil aggregates, which comprises a PAR-1 antagonist, a pharmaceutical salt thereof, or a solvate thereof.

### EXAMPLES

Below, the invention will be described in more detail with reference to the following examples and preparation examples. However, the present invention is not limited to these examples, which are provided only for the purpose of full disclosure of the present invention to those skilled in the art. It is not meant or even implied that the experiments described herein are all or only one experiment actually carried out. Although efforts have been made to guarantee the accuracy of the numerical values used herein (e.g., volume, temperature, concentration, etc.), experimental errors and deviations are considered to some extent.

The disclosure uses the abbreviations shown in Table 1 as follows:

TABLE 1

| Abbreviation | Description |
|---|---|
| E5555 | Proprietary PAR-1 antagonist |
| ASA | Acetylsalicylic acid, aspirin |
| CAD | Coronary artery disease |
| ACS | Acute coronary syndromes |
| PAU | Platelet activation units |
| MFI | Mean fluorescence intensity |
| PRP | Platelet rich plasma |
| PPP | Platelet poor plasma |
| QR | Quorum Review |
| VTNR | Vitronectin Receptor |
| LAMP | Lysosome associated membrane protein |
| PMA | Platelet-monocyte aggregates |
| ADP | Adenosine diphosphate |
| TRAP | Thrombin Receptor Activating Peptide |

This study attempts to determine the in vitro effects of E5555 on unstimulated, and TRAP-stimulated platelet aggregation, rapid analyzers readings, and surface receptor expression in human volunteers, and patients with CAD. Secondary Objective: To define a dose-dependency (if any) of E5555 properties on top of ASA or/and clopidogrel on platelet activity including subclinical, maintenance, loading doses. Concept: E5555 in vitro will significantly and dose-dependently inhibit platelet expression of surface receptors.

The methodology employed was conventional aggregation induced by 5 μM ADP, 1 μg/ml collagen, 10 μM TRAP, whole blood aggregation with 1 μg/ml collagen, Ultegra, and PFA-100 Analyzers, expression of 14 intact, and TRAP-stimulated major receptors by whole blood flow cytometry were utilized to assess platelet activity after preincubation with escalating concentrations of E5555 (20 ng/ml, 50 ng/ml, and 100 ng/ml). The experiments were performed in healthy volunteers, CAD patients treated with ASA, and CAD patients treated with ASA and clopidogrel combination (n=10, for each group).

E5555 is a compound having the below structure and also known as 1-(3-tert-butyl-4-methoxy-5-morpholinophenyl)-2-(5,6-diethoxy-7-fluoro-1-imino-1,3-dihy dro-2H-isoindol-2-yl)ethanone hydrobromide, which has a molecular weight of 608.54 as the bromide salt. E5555 is a first small molecule that acts as a PAR-1 antagonist. E5555 can be produced according to the method(s) described in WO02/085855.

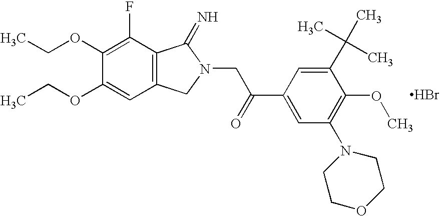

Blood samples for platelet studies were obtained from 30 individuals: (i) healthy volunteers (n=10) undergoing employment drug screening at St. Joseph Hospital of Baltimore; (ii) patients with documented CAD treated with ASA (81 mg/daily); and (iii) patients with documented CAD treated with ASA (81 mg/daily) in combination with clopidogrel (75 mg/daily).

An Informed Consent was obtained from all volunteers. Drug users were excluded from data analysis. The study was approved by Quorum Institutional Review Board. All subjects underwent blood sampling after at least 30 minutes of rest and two or more hours of fasting. Blood (28 ml) was drawn between 8 and 10 a.m. in order to avoid any diurnal influence and sampled from an antecubital vein using a 21-gauge butterfly needle containing 3.2% sodium citrate (1:9 volume) after having discarded the first 1.5 ml of free running blood. Four Vacutainer tubes (7.0 ml) were collected for a total of 28 ml of whole blood-citrate mixture from each study participant. Chronologically, blood samples were delivered to the core laboratory facility within 45 minutes after drawing. Then all tubes of blood-citrate mixture were pooled. One portion of pooled blood (5 ml) was kept as an internal control and pretreated with the vehicle. The remaining blood (23 ml) was used for the in vitro exposure to E5555 at concentrations of 20 ng/ml, 50 ng/ml, and 100 ng/ml. The maximum time between the blood draw and exposure to E5555 did not exceed 1 hour. The platelet aggregation studies, Rapid Analyzers measures, and flow cytometry preparations were initiated within no more than 110 minutes following the blood draw, incubation with the drug, and preparation of the samples.

Fresh stock solutions of E5555 were prepared ex tempore on the same morning of the day on which the platelet studies were performed. E5555 was dissolved in ethanol (96%) and diluted with TBS-buffer to prepare a stock solution of 10 mM (final ethanol concentration: 0.0006%).

Platelet counts were determined in each PRP sample with a Coulter Counter ZM (Coulter Co., Hialeah, Fla.). Platelet numbers were adjusted to 3.50×108/ml for aggregation using homologous platelet-poor plasma. Platelet aggregation was stimulated with 5 μM ADP, 1 μg/ml collagen, or 10 μM TRAP. All agonists were obtained from Chronolog Corporation (Havertown, Pa.) and Sigma (St. Louis, Miss.). Platelet aggregation experiments were performed using a Chrono-Log Lumi-Aggregometer (model 560-Ca). Aggregation curves were recorded for 4 minutes and analyzed according to internationally established standards using Aggrolink® software. Platelet aggregability was expressed as the percentage of light transmittance change from baseline using platelet-poor plasma as a reference.

Whole blood aggregation was determined using a Chronolog device 592A. The whole blood-citrate mixture was diluted 1:1 with 0.5 ml phosphate buffered saline and gently swirled. The sample then was allowed to warm to 37° C. for 5 minutes then transferred to the assay well. The electrode was then placed in the cuvette and the sample was stimulated with 1 μg/ml collagen. The change in electrical impedance was digitally recorded. Each whole blood sample was analyzed utilizing 2 different clinical devices:
(1) a Rapid Platelet-Function Assay (Ultegra®) (Accumetrics, Inc., San Diego, Calif.), which is a rapid platelet-function assay aspirin cartridge test with TRAP used as agonist. Polystyrene beads coated with fibrinogen are placed in a cartridge along with the peptide that activates thrombin receptor. Anticoagulated whole blood was added to the cartridge, and agglutination between platelets and coated beads was recorded. The data mirrors turbidimetric platelet aggregation and presumably reflects platelet glycoprotein IIb/IIIa blockade. Ultegra RPFA-TRAP Assay results were reported as Platelet Activation Units (PAU); and
(2) Platelet-Function Analyzer PFA-100 ™ (Dade Behring, Deerfield Ill.), which is a high shear-inducing analyzer that simulates primary hemostasis after injury to a small vessel under flow conditions. The device provides a constant negative pressure that aspirates whole blood, which comes into contact with the collagen, coated membrane and then passes through the aperture. The time required to obtain occlusion of the aperture is digitally recorded. The time to aperture occlusion (the closure time) is recorded in seconds and is inversely related to the degree of shear-induced platelet activation. We used ADP/Collagen cartridges in the index study. Both PFA-100 and Ultegra assays were performed in duplicate. An electronic quality control test was performed on each instrument daily prior to performing any patient samples.

For whole blood flow cytometry (unstimulated), one ml of the blood citrate mixture was diluted with TBS buffer (10 mM Tris, 0.15 M NaCl, pH 7.4) and the following fluorescein isothiocyanate (FITC) conjugated monoclonal antibodies: CD 41 antigen (GP IIb) CD 42b (GP Ib), CD 62p (P-selectin); PAC-1 (GP IIb/IIIa activity), CD 31 (platelet/endothelial cell adhesion molecule [PECAM]-1), CD 51/CD 61 (vitronectin receptor: VTNR), CD 63 (LIMP or LAMP-3), CD 107a (LAMP-1), CD 151 (PETA-3), CD154 (CD40-ligand), CD 165 (GP37); CD36 (thrombospondin receptor, GPIV), G-coupled PAR-1/PAR-4 thrombin receptors (active and intact epitopes). Formation of platelet-leukocyte aggregates was assessed by dual labeling with pan-platelet marker (CD151), and then with CD14, the macrophage receptor for endotoxin lipopolysaccharides (PharMingen, and Coulter, San Diego, Calif., USA). The tubes were then covered with aluminum foil and incubated at 37° C. for 40 minutes with gentle rotation. After incubation, samples were immediately analyzed on a Becton Dickinson FACScan flow cytometer. The instrument was set up to measure forward light scatter, and FITC fluorescence. All parameters were collected using four-decade logarithmic amplification. The data was retrieved in list mode files and then analyzed. P selectin was expressed as percent positive cells. Other antigens were expressed as log mean fluorescence intensity.

For flow cytometry (TRAP-stimulated) all of the above flow cytometry experiments were identically repeated utilizing prestimulation with 10 μmol/L TRAP for 5 minutes at 37° C. To determine statistics, a Paired Sign Test was used to define significance of differences between vehicle and E5555-treated groups. The Mann-Whitney U test was used to analyze nonparametric data. Normally distributed data were expressed as mean±standard error (m±SE), and skewed data as median (range). All P-values were two sided. All analysis was performed using the SPSS v9.0 program (SPSS Inc., Chicago, Ill.).

The results for voluntary study participants were:

TABLE 2
Demographics and risk factors in healthy subjects.

| Variables | Volunteers (n = 10) |
|---|---|
| Females, n (%) | 5 (50%) |
| Age, mean ± SD | 29.2 ± 4.1 |
| Race | |
| Caucasian | 7 (70%) |
| African American | 3 (30%) |
| Risk Factors | |
| History of smoking | 7 (70%) |
| Obesity | 3 (30%) |
| Sedentary Life Style | 8 (80%) |
| Family History of CV diseases | 4 (40%) |

Males and females were included equally in the study. The majority of study participants were Caucasians, however, some African-Americans were also included. Among the risk factors for the development of vascular disease, smoking and sedentary lifestyle were common in healthy subjects, while obesity was not dominant. None of the study participants received anti-platelet agents including aspirin or clopidogrel for at least 2 weeks prior to the blood collection.

TABLE 3
Demographics, risk factors, and concomitant medications in CAD
patients treated with Aspirin, or Aspirin + Clopidogrel.

| | ASA | Aspirin + Clopidogrel |
|---|---|---|
| Variable | (n = 10) | (n = 10) |
| Age, years | 54.4 ± 8.2 | 57.2 ± 9.9 |
| Male | 7 (70%) | 8 (80%) |
| Ethnic origin | | |
| Caucasian | 6 (60%) | 6 (60%) |
| African-American | 2 (20%) | 1 (10%) |
| Hispanic | 1 (10%) | 1 (10%) |
| Asian | 1 (10%) | 2 (20%) |
| Risk Factors | | |
| Family History | 8 (80%) | 6 (60%) |
| Sedentary Life Style | 8 (80%) | 7 (70%) |
| Diabetes | 4 (40%) | 3 (30%) |
| Obesity | 6 (60%) | 5 (50%) |
| Current or past smoking | 6 (60%) | 7 (70%) |
| Hypertension | 7 (70%) | 5 (50%) |
| Hypercholesterolemia | 8 (80%) | 8 (80%) |
| PCI | 4 (40%) | 10 (100%) |
| Medications | | |
| ASA | 10 (100%) | 10 (100%) |
| Clopidogrel | 0 | 10 (100%) |
| ACE-inhibitors | 4 (40%) | 3 (30%) |
| ARB's | 3 (30%) | 5 (50%) |
| Beta-blockers | 2 (20%) | 1 (10%) |
| Ca-channel blockers | 1 (10%) | 2 (20%) |
| Statins | 9 (90%) | 8 (80%) |
| Anti-diabetic agents | 4 (40%) | 3 (30%) |
| Antidepressants | 3 (30%) | 4 (40%) |
| Proton pump inhibitors | 2 (20%) | 1 (10%) |

Samples were obtained from 10 patients treated with ASA, and 10 patients treated with ASA+clopidogrel combination patients chronically treated with clopidogrel all of whom underwent coronary stenting. Study donors were predominantly Caucasians, with a fairly evenly distributed male/female ratio. Majority of the participants exhibited positive family history of coronary artery disease, obesity, current or past history of smoking, and sedentary life-style. There was a high prevalence of hypertension and hypercholesterolemia, while diabetes was not dominant. Statins and anti-hypertensive agents where the most common concomitant medications and minority of patients were treated with antidepressants, and proton pump inhibitors.

Effects on platelet aggregation by E5555 are set forth in the following Tables:

TABLE 4
Platelet aggregation in healthy volunteers (n = 10).

| | E5555 concentration/Platelet Aggregation (%) | | | | |
|---|---|---|---|---|---|
| | Vehicle | 20 ng/ml | 50 ng/ml | 100 ng/ml | Ref. Range |
| Agonist | | | | | |
| ADP 5 μM§ | 77.3 ± 6.2 | 57.0 ± 9.2* | 59.4 ± 7.9* | 60.8 ± 10.0* | 60-87 |
| Collagen 1 μg/mL§ | 88.5 ± 4.8 | 67.6 ± 6.2* | 69.1 ± 6.5* | 72.8 ± 7.5* | 68-93 |
| Whole Blood | | | | | |
| Collagen 1 μg/mL§† | 24 ± 6 | 22 ± 5 | 25 ± 7 | 22 ± 6 | 19-27 |
| TRAP 10 μM§ | 93.7 ± 3.3 | 2.1 ± 1.2* | 1.3 ± 1.2* | 2.7 ± 1.8* | 78-97 |

Mean ± SD,
§p < 0.05 (Overall) Friedman's test;
†ohms;
*p < 0.05 (Paired) Sign Test compared to vehicle.

Pretreatment of blood samples with E5555 at escalating concentrations of 20, 50, and 100 ng/ml resulted in modest (15-20%), but significant inhibition of conventional platelet aggregation induced by ADP, and collagen. TRAP-induced aggregation was inhibited completely. Within the chosen concentrations of E5555, platelet aggregation was not inhibited a dose-dependent manner. The lowest blood level of the E5555 (20 ng/ml) was sufficient to produce the inhibitory effect to the full extend. Conventional PRP aggregometry is more sensitive than whole blood impedance aggregometry in detecting the in vitro effects of E5555.

TABLE 5
Platelet aggregation in CAD patients treated with ASA (n = 10)

| | E5555 concentration/Platelet Aggregation (%) | | | | |
|---|---|---|---|---|---|
| | Vehicle | 20 ng/ml | 50 ng/ml | 100 ng/ml | Ref. Range |
| Agonist | | | | | |
| ADP 5 μM§ | 63.0 ± 9.7 | 51.8 ± 4.7* | 53.3 ± 5.9* | 56.2 ± 5.3 | 60-87 |
| Collagen 1 μg/mL§ | 34.1 ± 5.4 | 23.9 ± 4.8* | 26.1 ± 4.0* | 25.7 ± 5.2* | 68-93 |
| Whole Blood | | | | | |
| Collagen 1 μg/mL§† | 15 ± 4 | 11 ± 4 | 14 ± 6 | 12 ± 5 | 19-27 |
| TRAP 10 μM§ | 74.2 ± 6.8 | 2.7 ± 1.4* | 3.3 ± 1.8* | 2.9 ± 2.0* | 78-97 |

Mean ± SD,
§p < 0.05 (Overall) Friedman's test;
†ohms;
*p < 0.05 (Paired) Sign Test compared to vehicle

In the ASA-treated patients with documented coronary artery disease, E5555 exhibit mild (10-12%) inhibition of platelet activity when aggregation was induced by ADP, or collagen, consistently and fully inhibiting TRAP-induced aggregation.

TABLE 6
Platelet aggregation in CAD patients treated with ASA + clopidogrel (n = 10).

| | E5555 concentration/Platelet Aggregation (%) | | | | |
|---|---|---|---|---|---|
| | Vehicle | 20 ng/ml | 50 ng/ml | 100 ng/ml | Ref. Range |
| Agonist | | | | | |
| ADP 5 μM | 37.2 ± 5.9 | 40.8 ± 7.1 | 42.6 ± 8.3 | 39.3 ± 5.1 | 60-87 |
| Collagen 1 μg/mL,§ | 30.8 ± 7.0 | 20.6 ± 3.2* | 24.0 ± 3.3* | 21.7 ± 4.2* | 68-93 |
| Whole Blood | | | | | |
| Collagen 1 μg/mL§† | 16 ± 5 | 13 ± 5 | 12 ± 7 | 14 ± 4 | 19-27 |
| TRAP 10 μM§ | 62.9 ± 8.6 | 2.2 ± 1.6* | 1.8 ± 1.0* | 2.7 ± 2.2* | 78-97 |

Mean ± SD,
§p < 0.05 (Overall) Friedman's test;
†ohms;
*p < 0.05 (Paired) Sign Test compared to vehicle

In CAD patients treated with combination (ASA+clopidogrel) antiplatelet therapy, ADP-induced aggregation remains unchanged, moderate inhibition of collagen-induced aggregation, and profound blockade of TRAP-induced aggregation were confirmed.

TABLE 7
Ultegra Platelet Analyzer.

| | E5555 concentration/Platelet Activation Units (PAU) | | | | |
|---|---|---|---|---|---|
| Group | Vehicle | 20 ng/ml | 50 ng/ml | 100 ng/ml | Ref. Range |
| Volunteers§ | 172 ± 33 | 270 ± 30* | 126 ± 20* | 117 ± 21* | 150-215 |
| Patients (ASA)§ | 165 ± 17 | 118 ± 12* | 114 ± 11* | 113 ± 14* | 150-215 |
| Patients (ASA + C)§ | 143 ± 16 | 115 ± 21* | 108 ± 20* | 112 ± 18* | 150-215 |

Mean ± SD,
§p < 0.05 (Overall) Friedman's test;
*p < 0.05 (Paired) Sign Test compared to vehicle

Ultegra Analyzer data reveal reduction of PAU after E5555 most prominent in volunteers, but also significant in CAD patients treated with ASA alone, or in combination with clopidogrel. Lack of dose-dependent response was also observed.

TABLE 8
Platelet Function Analyzer (PFA-100).

| | E5555 concentration/Closure time (s) | | | | |
|---|---|---|---|---|---|
| Group | Vehicle | 20 ng/ml | 50 ng/ml | 100 ng/ml | Ref. Range |
| Volunteers§ | 218 ± 28 | >300*† | >300*† | >300*† | 150-240 |
| Patients (ASA)§ | 255 ± 34 | >300*† | >300*† | >300*† | 150-240 |
| Patients (ASA + C)§ | 280 ± 30† | >300*† | >300*† | >300*† | 150-240 |

Mean ± SD,
§p < 0.05 (Overall) Friedman's test;
*p < 0.05 (Paired) Sign Test compared to vehicle;
†the instrument readings exceeds the recording time of 5 minutes (300 s), the less shear induced platelet activity, the longer is the closure time. The PFA-100 device is not suitable for the precise determination of in vitro antiplatelet properties of E5555 due to the upper limit of the closure time prolongation.

TABLE 9
Unstimulated Whole Blood Flow Cytometry.

| | E5555 concentration/Mean Fluorescence Intensity (MFI) | | | | |
|---|---|---|---|---|---|
| Group | Vehicle | 20 ng/ml | 50 ng/ml | 100 ng/ml | Ref. Range |
| PECAM-1 (CD31) | | | | | |
| Volunteers§ | 76 ± 11 | 64 ± 10* | 60 ± 8* | 64 ± 11* | 65-88 |
| Patients (ASA)§ | 72 ± 14 | 52 ± 9* | 53 ± 7* | 57 ± 10* | 65-88 |
| Patients (ASA + C)§ | 73 ± 12 | 55 ± 11* | 51 ± 9* | 54 ± 8* | 65-88 |
| GPIIb antigen (CD41) | | | | | |
| Volunteers§ | 487 ± 42 | 423 ± 27* | 409 ± 25* | 416 ± 24* | 360-522 |
| Patients (ASA)§ | 488 ± 51 | 391 ± 44* | 404 ± 31* | 396 ± 42* | 360-522 |
| Patients (ASA + C)§ | 362 ± 39 | 219 ± 25* | 240 ± 32* | 233 ± 30* | 360-522 |
| GPIb (CD42b) | | | | | |
| Volunteers§ | 203 ± 18 | 162 ± 19* | 145 ± 20* | 157 ± 16* | 160-260 |
| Patients (ASA)§ | 141 ± 12 | 106 ± 5* | 109 ± 7* | 111 ± 10* | 160-260 |
| Patients (ASA + C)§ | 152 ± 14 | 103 ± 7* | 94 ± 12* | 100 ± 15* | 160-260 |
| PAC-1 (GPIIb/IIIa) | | | | | |
| Volunteers§ | 12.4 ± 1.8 | 10.7 ± 1.3* | 9.2 ± 1.5* | 9.9 ± 2.0* | 8-14 |
| Patients (ASA)§ | 13.0 ± 2.1 | 10.4 ± 1.2* | 10.1 ± 2.0* | 11.7 ± 2.1 | 8-14 |
| Patients (ASA + C)§ | 8.3 ± 1.4 | 5.9 ± 0.8* | 6.2 ± 1.0* | 6.3 ± 0.7* | 8-14 |
| VTNR (CD51/61) | | | | | |
| Volunteers§ | 10.4 ± 0.9 | 8.7 ± 0.6* | 9.1 ± 1.0* | 9.6 ± 1.1 | 7-12 |
| Patients (ASA)§ | 10.6 ± 2.3 | 8.4 ± 1.5* | 8.6 ± 2.1* | 8.3 ± 14* | 7-12 |
| Patients (ASA + C)§ | 11.0 ± 2.8 | 7.9 ± 1.8* | 8.2 ± 1.6* | 9.0 ± 2.4 | 7-12 |
| P-selectin (CD62p) † | | | | | |
| Volunteers | 11.0 ± 1.7 | 10.8 ± 2.0 | 12.3 ± 2.2 | 11.4 ± 1.8 | 7-13% |
| Patients (ASA) | 9.6 ± 1.3 | 9.7 ± 1.5 | 10.1 ± 1.8 | 10.7 ± 1.6 | 7-13% |
| Patients (ASA + C) | 7.4 ± 0.8 | 7.6 ± 2.0 | 7.7 ± 2.2 | 8.0 ± 11.5 | 7-13% |
| LAMP-3 (CD63) | | | | | |
| Volunteers | 7.8 ± 1.3 | 8.1 ± 2.0 | 8.0 ± 1.7 | 7.7 ± 2.1 | 6-10 |
| Patients (ASA) | 6.8 ± 1.4 | 7.0 ± 1.6 | 7.2 ± 1.5 | 6.5 ± 1.9 | 6-10 |
| Patients (ASA + C) | 7.0 ± 1.1 | 7.2 ± 1.3 | 7.0 ± 1.7 | 6.9 ± 1.8 | 6-10 |
| LAMP-1 (CD107a) | | | | | |
| Volunteers | 4.4 ± 0.6 | 4.7 ± 0.5 | 4.6 ± 0.6 | 4.4 ± 1.0 | 4-6 |
| Patients (ASA) | 4.7 ± 0.8 | 4.3 ± 1.2 | 5.0 ± 1.7 | 4.3 ± 1.5 | 4-6 |
| Patients (ASA + C) | 4.5 ± 1.1 | 4.2 ± 1.6 | 4.7 ± 1.3 | 4.8 ± 1.4 | 4-6 |
| PMA (CD14 + CD151) | | | | | |
| Volunteers§ | 153 ± 18 | 130 ± 16* | 128 ± 18* | 124 ± 23* | 90-170 |
| Patients (ASA)§ | 140 ± 27 | 114 ± 18* | 114 ± 19* | 107 ± 20* | 90-170 |
| Patients (ASA + C) | 108 ± 19 | 104 ± 16 | 100 ± 27 | 105 ± 21 | 90-170 |
| CD-40L (CD154) | | | | | |
| Volunteers | 5.2 ± 0.6 | 5.4 ± 0.8 | 5.3 ± 0.5 | 5.0 ± 0.9 | 3.8-7.2 |
| Patients (ASA) | 5.4 ± 0.9 | 5.0 ± 1.0 | 5.2 ± 0.8 | 5.1 ± 1.0 | 3.8-7.2 |
| Patients (ASA + C) | 5.0 ± 1.1 | 5.1 ± 1.0 | 5.2 ± 0.7 | 5.3 ± 1.3 | 3.8-7.2 |
| GP37 (CD165) | | | | | |
| Volunteers | 30 ± 6 | 27 ± 8 | 28 ± 7 | 26 ± 6 | 24-37 |
| Patients (ASA) | 32 ± 10 | 30 ± 7 | 27 ± 12 | 28 ± 11 | 24-37 |
| Patients (ASA + C) | 30 ± 9 | 25 ± 11 | 28 ± 10 | 25 ± 12 | 24-37 |
| Thrombospondin- | | | | | |
| Receptor (CD36) | | | | | |
| Volunteers§ | 11.3 ± 2.0 | 8.4 ± 1.1* | 8.2 ± 0.8* | 8.4 ± 1.0* | 8-12 |
| Patients (ASA)§ | 9.6 ± 1.3 | 7.2 ± 0.5* | 7.0 ± 0.7* | 7.4 ± 0.9* | 8-12 |
| Patients (ASA + C) | 6.7 ± 1.5 | 6.8 ± 1.9 | 7.0 ± 1.0 | 7.1 ± 1.2 | 8-12 |
| WEDE15 | | | | | |
| Volunteers | 43 ± 8 | 40 ± 9 | 41 ± 6 | 40 ± 10 | 26-54 |
| Patients (ASA) | 37 ± 9 | 40 ± 11 | 38 ± 8 | 35 ± 5 | 26-54 |
| Patients (ASA + C) | 28 ± 5 | 30 ± 8 | 27 ± 6 | 29 ± 12 | 26-54 |
| SPAN-12 | | | | | |
| Volunteers§ | 25 ± 6 | 19 ± 4* | 19 ± 6* | 18 ± 6* | 20-30 |
| Patients (ASA)§ | 28 ± 7 | 14 ± 3* | 11 ± 5* | 13 ± 5* | 20-30 |
| Patients (ASA + C)§ | 20 ± 9 | 11 ± 4* | 10 ± 6* | 12 ± 7* | 20-30 |

Mean ± SD,
§p < 0.05 (Overall) Friedman's test;
*p < 0.05 (Paired) Sign Test compared to vehicle;
† measured in % positive cells

TABLE 10
TRAP-stimulated Whole Blood Flow Cytometry.

| | E5555 concentration/Mean Fluorescence Intensity (MFI) | | | | |
|---|---|---|---|---|---|
| Group | Vehicle | 20 ng/ml | 50 ng/ml | 100 ng/ml | Ref. Range |
| PECAM-1 (CD31) | | | | | |
| Volunteers§ | 72 ± 8 | 60 ± 9* | 55 ± 10* | 60 ± 7* | None |
| Patients (ASA)§ | 77 ± 11 | 50 ± 6* | 54 ± 9* | 52 ± 12* | Available |
| Patients (ASA + C)§ | 80 ± 9 | 58 ± 9* | 60 ± 7* | 57 ± 11* | |
| GPIIb antigen (CD41) | | | | | |
| Volunteers§ | 618 ± 39 | 504 ± 18* | 390 ± 31* | 364 ± 37* | |
| Patients (ASA)§ | 645 ± 47 | 471 ± 38* | 376 ± 35* | 304 ± 40* | |
| Patients (ASA + C)§ | 501 ± 55 | 328 ± 41* | 209 ± 30* | 176 ± 19* | |
| GPIb (CD42b) | | | | | |
| Volunteers§ | 246 ± 15 | 134 ± 17* | 125 ± 24* | 150 ± 26* | |
| Patients (ASA)§ | 208 ± 23 | 102 ± 9* | 104 ± 17* | 100 ± 14* | |
| Patients (ASA + C)§ | 219 ± 27 | 105 ± 14* | 102 ± 19* | 137 ± 18* | |
| PAC-1 (GPIIb/IIIa) | | | | | |
| Volunteers§ | 105 ± 9 | 68 ± 8* | 17 ± 9* | 7 ± 3* | |
| Patients (ASA)§ | 80 ± 11 | 52 ± 6* | 9 ± 3* | 4 ± 1* | |
| Patients (ASA + C)§ | 62 ± 7 | 41 ± 5* | 9 ± 1* | 5 ± 2* | |
| VTNR (CD51/61) | | | | | |
| Volunteers | 9.3 ± 0.7 | 10.4 ± 1.3 | 10.1 ± 1.7 | 10.7 ± 1.5 | |
| Patients (ASA) | 10.1 ± 2.0 | 10.8 ± 2.1 | 10.5 ± 1.3 | 9.9 ± 1.3 | |
| Patients (ASA + C) | 9.7 ± 1.8 | 10.2 ± 1.5 | 10.9 ± 1.6 | 11.0 ± 2.0 | |
| P-selectin (CD62p) † | | | | | |
| Volunteers§ | 57 ± 4 | 31 ± 8* | 14 ± 3* | 11 ± 3* | |
| Patients (ASA)§ | 50 ± 7 | 22 ± 6* | 15 ± 4* | 8 ± 4* | |
| Patients (ASA + C)§ | 27 ± 7 | 11 ± 5* | 8 ± 3* | 3 ± 1* | |
| LAMP-3 (CD63) | | | | | |
| Volunteers§ | 49 ± 9 | 26 ± 7* | 8 ± 2* | 7 ± 2* | |
| Patients (ASA)§ | 44 ± 10 | 24 ± 8* | 9 ± 3* | 5 ± 1* | |
| Patients (ASA + C)§ | 46 ± 7 | 20 ± 5* | 5 ± 3* | 4 ± 1* | |
| LAMP-1 (CD107a) | | | | | |
| Volunteers | 7.4 ± 1.0 | 5.9 ± 0.8* | 4.9 ± 0.4* | 4.7 ± 0.8* | |
| Patients (ASA) | 7.8 ± 0.9 | 4.1 ± 0.7* | 4.0 ± 0.5* | 3.1 ± 0.7* | |
| Patients (ASA + C) | 7.1 ± 0.6 | 4.0 ± 0.8* | 3.2 ± 1.0* | 3.0 ± 1.1* | |
| PMA (CD14 + CD151) | | | | | |
| Volunteers§ | 151 ± 27 | 133 ± 19* | 118 ± 14* | 125 ± 20* | |
| Patients (ASA)§ | 144 ± 22 | 109 ± 23* | 82 ± 10* | 116 ± 23* | |
| Patients (ASA + C)§ | 160 ± 30 | 100 ± 21* | 77 ± 23* | 108 ± 19* | |
| CD-40L (CD154) | | | | | |
| Volunteers§ | 7.2 ± 0.5 | 6.3 ± 0.6* | 5.7 ± 0.6* | 5.7 ± 0.4* | |
| Patients (ASA)§ | 7.0 ± 0.8 | 5.2 ± 0.4* | 4.8 ± 0.5* | 5.0 ± 0.8* | |
| Patients (ASA + C)§ | 7.7 ± 0.6 | 4.9 ± 0.5* | 4.0 ± 0.3* | 4.7 ± 0.5* | |
| GP37 (CD165) | | | | | |
| Volunteers§ | 41 ± 9 | 32 ± 6* | 28 ± 5* | 29 ± 4* | |
| Patients (ASA)§ | 40 ± 7 | 27 ± 5* | 20 ± 7* | 22 ± 6* | |
| Patients (ASA + C)§ | 38 ± 10 | 27 ± 4* | 18 ± 4* | 21 ± 9* | |
| Thrombospondin- | | | | | |
| Receptor (CD36) | | | | | |
| Volunteers§ | 14 ± 5 | 9 ± 1* | 9 ± 2* | 9 ± 1* | |
| Patients (ASA)§ | 14 ± 3 | 6 ± 2* | 7 ± 1* | 7 ± 2* | |
| Patients (ASA + C)§ | 12 ± 4 | 6 ± 1* | 5 ± 1* | 6 ± 2* | |
| WEDE15 | | | | | |
| Volunteers | 39 ± 6 | 40 ± 7 | 40 ± 5 | 40 ± 8 | |
| Patients (ASA) | 40 ± 7 | 38 ± 6 | 39 ± 6 | 40 ± 8 | |
| Patients (ASA + C) | 32 ± 3 | 33 ± 5 | 34 ± 6 | 33 ± 6 | |
| SPAN-12 | | | | | |
| Volunteers§ | 27 ± 5 | 20 ± 3* | 17 ± 5* | 17 ± 4* | |
| Patients (ASA)§ | 27 ± 6 | 12 ± 4* | 9 ± 4* | 10 ± 6* | |
| Patients (ASA + C)§ | 22 ± 4 | 9 ± 2* | 9 ± 3* | 11 ± 3* | |

Mean ± SD,
§p < 0.05 (Overall) Friedman's test;
*p < 0.05 (Paired) Sign Test Compared to vehicle;
† measured in % positive cells.

E5555, at concentrations mimicking human therapeutic range, inhibits various platelet biomarkers. Platelet inhibition is usually mild, exhibited already for the lowest E5555 concentration, and is not dose-dependent without TRAP stimulation. E5555 causes 10-15% inhibition of ADP— and collagen-induced platelet aggregation in plasma, TRAP-induced aggregation was inhibited almost completely. Platelet Analyzer readings revealed reduced shear-induced activation and diminished fibrinogen binding with E5555 treatment. These data were confirmed by significant reduction of PECAM-1, GP IIb/IIIa antigen, and activity with PAC-1, GPIb, thrombospondin receptor, and vitronectin receptor expression. Formation of platelet-monocyte aggregates was also significantly reduced by E5555. After TRAP stimulation, dose-dependent effect between 20 ng/ml and 50 ng/ml E5555 doses has been observed. P selectin, LAMP-1, LAMP, and CD40-ligand were not affected by E5555.

E5555 in vitro mildly, but significantly inhibits platelet activity beyond PAR-1 blockade in vitro. Antiplatelet potency of ASA and clopidogrel are enhanced by E5555 providing rationale for the synergic use. The clinical relevance of the observed in vitro changes is encouraging. Inhibition of the production of GPIb, GPIIb/IIIa, PECAM-1, thrombospondin receptor, and vitronectin receptor inhibition suggests unique vascular protective properties of PAR-1 antagonists exemplified by E5555 in addition to the current antithrombotic regimens.

The above description and drawings are to illustrate certain embodiments of the present invention. It is not intended that the present invention be limited to these versions, but only by the appended claims. What is desired of protection by U.S. Letters Patent is set forth as follows:

1. A method of treating a vascular disease in an individual, comprising:
selecting an individual having an elevated level of glycoprotein Ib, glycoprotein IIb/IIIa, PECAM-1, vitronectin receptor or thrombospondin receptor, or formation of platelet-neutrophil aggregates; and
administering a therapeutically effective amount of a PAR-1 antagonist, a pharmaceutical salt thereof, or a solvate thereof to the individual.

2. The method of claim 1, wherein said individual is also selected based on the individual having a PAR-1 platelet expression in a normal range.

3. The method of claim 1, wherein the vascular disease is selected from the group consisting of coronary artery disease, myocardial infarction, angina, stroke, pulmonary embolism, transient ischemic attack, deep vein thrombosis, thrombotic re-occlusion subsequent to a coronary intervention procedure, heart surgery, vascular surgery, peripheral vascular thrombosis, Syndrome X, heart failure, and a disease in which a narrowing of at least one coronary artery occurs.

4. The method of claim 1, wherein the PAR-1 antagonist is 1-(3-tert-butyl-4-methoxy-5-morpholinophenyl)-2-(5,6-diethoxy-7-fluoro-1-imino-1,3-dihy dro-2H-isoindol-2-yl)ethanone hydrobromide or a metabolite thereof.

5. The method of claim 4, wherein the PAR-1 antagonist is administered orally in a therapeutically effective amount that is between about 0.1 mg and about 500 mg per day.

6. The method of claim 1, wherein the elevated level of PAR-1, glycoprotein Ib, glycoprotein IIb/IIIa, PECAM-1, vitronectin receptor or thrombospondin receptor, or formation of neutrophil aggregates is found on platelets.

7. The method of claim 1, wherein the PAR-1 antagonist is administered orally, intravenously, intramuscularly, subcutaneously, parenterally, nasally, by inhalation, by implant, or by suppository.

8. The method of claim 1, wherein the method further comprises administering a second compound selected from the group consisting of an anti-platelet compound, an antithrombotic, an anticoagulant, or a thrombolytic agent.

9. The method of claim 1, wherein the individual is a human.

10. A method of treating a vascular disease in an individual, comprising:
assessing a level of glycoprotein Ib, glycoprotein IIb/IIIa, PECAM-1, vitronectin receptor or thrombospondin receptor, or formation of platelet-neutrophil aggregates in the individual, and comparing said level or formation of said aggregates to a control to determine if an elevated level of glycoprotein Ib, glycoprotein IIb/IIIa, PECAM-1, vitronectin receptor or thrombospondin receptor, or formation of platelet-neutrophil aggregates exists; and
if an elevated level of glycoprotein Ib, glycoprotein IIb/IIIa, PECAM-1, vitronectin receptor or thrombospondin receptor, or formation of platelet-neutrophil aggregates exists, administering a therapeutically effective amount of a PAR-1 antagonist, a pharmaceutical salt thereof, or a solvate thereof to the individual to reduce the level of glycoprotein Ib, glycoprotein IIb/IIIa, PECAM-1, vitronectin receptor or thrombospondin receptor, or formation of platelet-neutrophil aggregates to treat the vascular disease.

11. The method of claim 10, further comprising reducing the level of glycoprotein Ib, glycoprotein IIb/IIIa, PECAM-1, vitronectin receptor or thrombospondin receptor, or formation of platelet-neutrophil aggregates by at least 10%, as compared to the assessed level of glycoprotein Ib, glycoprotein IIb/IIIa, PECAM-1, vitronectin receptor or thrombospondin receptor, or assessed formation of platelet-neutrophil aggregates.

12. The method of claim 10, wherein the vascular disease is selected from the group consisting of coronary artery disease, myocardial infarction, angina, stroke, pulmonary embolism, transient ischemic attack, deep vein thrombosis, thrombotic re-occlusion subsequent to a coronary intervention procedure, heart surgery, vascular surgery, peripheral vascular thrombosis, Syndrome X, heart failure, and a disease in which a narrowing of at least one coronary artery occurs.

13. The method of claim 10, further comprising administering one or more of vascular disease treating drugs.

14. The method of claim 10, wherein the PAR-1 antagonist is 1-(3-tert-butyl-4-methoxy-5-morpholinophenyl)-2-(5,6-diethoxy-7-fluoro-1-imino-1,3-dihy dro-2H-isoindol-2-yl)ethanone hydrobromide or a metabolite thereof.

15. The method of claim 10, wherein the PAR-1 antagonist is administered orally, intravenously, intramuscularly, subcutaneously, parenterally, nasally, by inhalation, by implant, or by suppository.

16. The method of claim 10, wherein the method further comprises administering a second compound selected from the group consisting of an anti-platelet compound, an antithrombotic, an anticoagulant, or a thrombolytic agent.

17. A method of preventing a vascular disease in an individual, comprising:
selecting an individual having an elevated level of glycoprotein Ib, glycoprotein IIb/IIIa, PECAM-1, vitronectin receptor or thrombospondin receptor, or formation of platelet-neutrophil aggregates; and
administering a therapeutically effective amount of a PAR-1 antagonist, a pharmaceutical salt thereof, or a solvate thereof to the individual.

18. A method of preventing a vascular disease in an individual, comprising:
assessing a level of glycoprotein Ib, glycoprotein IIb/IIIa, PECAM-1, vitronectin receptor or thrombospondin receptor, or formation of platelet-neutrophil aggregates in the individual, and comparing said level or formation of said aggregates to a control to determine if an elevated level of glycoprotein Ib, glycoprotein IIb/IIIa, PECAM-1, vitronectin receptor or thrombospondin receptor, or formation of platelet-neutrophil aggregates exists; and
if an elevated level of glycoprotein Ib, glycoprotein IIb/IIIa, PECAM-1, vitronectin receptor or thrombospondin receptor, or formation of platelet-neutrophil aggregates exists, administering a therapeutically effective amount of a PAR-1 antagonist, a pharmaceutical salt thereof, or a solvate thereof to the individual.

19. A method of inhibiting the production of glycoprotein Ib, glycoprotein IIb/IIIa, PECAM-1, vitronectin receptor or thrombospondin receptor, or formation of platelet-neutrophil aggregates on a platelet in an individual who is at risk for developing a vascular disease, comprising administering to the individual a therapeutically effective amount of a PAR-1 antagonist, a pharmaceutical salt thereof, or a solvate thereof.

20. The method of claim 19, wherein the platelet is contacted with the PAR-1 antagonist, ex vivo.
